(19) United States
(12) Patent Application Publication
(10) Pub. No.: US 2008/0081998 A1
(43) Pub. Date: Apr. 3, 2008

(54) SYSTEM AND METHOD FOR THREE-DIMENSIONAL AND FOUR-DIMENSIONAL CONTRAST IMAGING

(75) Inventors: Lihong Pan, Brookfield, WI (US); Kirstin Nora LaConte, Waukesha, WI (US); Sharathchander Sirivolu, Waukesha, WI (US)

Correspondence Address: PETER VOGEL;GE HEALTHCARE, 3000 N. GRANDVIEW BLVD., SN-477, WAUKESHA, WI, 53188, US

(73) Assignee: GENERAL ELECTRIC COMPANY, Schenectady, NY (US)

(21) Appl. No.: 11/538,244

(22) Filed: Oct. 3, 2006

(51) Int. Cl. A61B 8/00

(52) U.S. Cl. 600/458

(57) ABSTRACT

Contrast-enhancing agents enhance 3D and/or 4D ultrasound imagery, by which ultrasound machines acquire ultrasound image data and processors convert the ultrasound image data into contrast-enhanced 3D and/or 4D images using the contrast-enhancing agents. Various other embodiments may also include CTI processors, which can be adapted to generate CTI image data, and/or TIC processors, which can be adapted to generate TIC image data. Systems, methods, and computer-readable storage medium including a set of instructions for a computer are disclosed.

### BACKGROUND OF INVENTION

The present invention generally relates to three-dimensional (3D) and four-dimensional (4D) contrast imaging in a healthcare environment. In particular, the present invention relates to a system and method for applying contrast imaging on 3D and 4D images using ultrasound technology to improve the accuracy and efficiency of a diagnosis. A user is able to view a patient's images based on a patient's images from previous examinations.

A clinical or healthcare environment is a crowded, demanding environment. Thus, applying contrast imaging on 3D and 4D images using ultrasound technology to improve the efficiency in which a user is able to view a patient's images based on a patient's images from current or previous examinations would be highly desirable. Further, the ability to more accurately diagnose a patient by using 3D and/or 4D contrast imaging is of great value to a medical practitioner and/or technician as well as the patient.

Ultrasound plays a vital role in obstetrical imaging as well as treating and/or detecting kidney stones, peripheral vascular disease, stenosis of the cartorid arteries (which may cause a stroke), and deep vein thrombosis (which may cause a stroke, coronary blockage or pulmonary embolism), among other things. Ultrasound is also used in image-guided interventions such as biopsies and drainages, for example.

Ultrasound technology uses high-frequency sound waves to visualize soft tissue structures in the body in real time. Ultrasound images had been viewed as static two dimensional (2D) images. However, with current technology, both 3D images and 3D images viewed in real-time (i.e., 4D images) are available.

3D and 4D imaging provides several advantages over 2D technology. For example, viewing ultrasound images in 3D and 4D can greatly enhance the diagnostic process by providing unique information and perspective for the diagnostic process. A significant reduction in inaccurate diagnosis can occur using 3D and 4D images because the clearer 3D and 4D pictures provide an easier understanding of complex structures. 3D and 4D technology coupled with volume data makes it possible to analyze the tissue concerned from multiple angles. Using 3D and 4D technology, coronal image plane and anatomical views not possible with 2D scanning can be obtained. Further, better qualitative and quantitative information can be obtained to diagnose effectively because 3D and 4D technology allows for a complete examination through increased perspective from volume data. Using 3D and 4D imaging, all planes of view are reproducible (i.e., a virtual patient). 3D and 4D technology may allow for reduced study time, decrease patient waiting times, and an overall faster examination procedure.

Additionally, 4D imaging may provide additional advantages over other 3D imaging diagnostic processes. 4D technology allows real-time diagnoses to be made on moving objects or organs. 4D imaging may allow for improved accuracy of the diagnosis of a fetus, for example, by permitting a medical practitioner and/or technician to view movement patterns of a fetus allowing conclusions to be drawn about their development. In ultrasound guided biopsies, 4D technology can greatly increase the accuracy of the diagnosis due to the full control of needle movements in real time in all three image planes.

3D and 4D ultrasound imaging technology is now available on many ultrasound systems. However, there would be a significant clinical benefit to applying 3D/4D technology using ultrasound contrast imaging. With contrast imaging, blood flow in very small blood vessel becomes visible. In contrast imaging, contrast agents are injected into the blood vessel through a vein. The contrast agents generally last only several minutes in the body. Contrast imaging is performed immediately after the injection. Contrast enhancement dynamic change is used to detect and classify a lesion. An ultrasound transducer needs to maintain on a lesion for the entire time when the contrast agents are in the body. 3D/4D contrast imaging allows the user to visualize the vascularity around a tumor since it captures the contrast information in the whole volume. It also makes it easier to maintain the ultrasound transducer on the lesion target. Contrast imaging using 3D and/or 4D images may allow a medical practitioner and/or technician to more easily identify tumors by monitoring the contrast enhancement.

3D and 4D technology is used by medical professionals to assist in visualizing and detecting abnormalities in a patient. Contrast imaging using 3D and 4D images may be necessary for medical professionals to properly, effectively, and efficiently diagnose patients. A system and method for applying contrast imaging on 3D and 4D images using ultrasound technology would improve the efficiency and accuracy of an ultrasound examination. Thus, there is a need for a system and method for applying contrast imaging on 3D and 4D images using ultrasound technology to improve the accuracy of a diagnosis and the efficiency in which a medical practitioner and/or technician is able to view a patient's images based on a patient's images from current or previous examinations.

### SUMMARY OF INVENTION

In certain embodiments of the inventive arrangements, ultrasound imaging systems comprise an ultrasound machine for acquiring ultrasound image data and a processor for converting the ultrasound image data into a contrast-enhanced 3D and/or 4D image. It may also further comprise a CTI processor, which can be adapted to generate CTI image data, and/or a TIC processor, which can be adapted to generate TIC image data.

In other embodiments, methods for acquiring ultrasound images comprise providing an ultrasound machine for acquiring ultrasound image data and providing a processor for converting the ultrasound image data into a 3D and/or 4D contrast-enhanced image. It may also further comprise providing a CTI processor, which can be adapted to generate CTI image data, and/or a TIC processor, which can be adapted to generate TIC image data.

In yet other embodiments, methods for acquiring 3D and/or 4D contrast-enhanced ultrasound images comprise providing a contrast-enhancing agent, providing an ultrasound machine for acquiring ultrasound image data, and providing a processor for converting the ultrasound image data into 3D and/or 4D contrast-enhanced images using the contrast-enhancing agent.

In still yet other embodiments, 3D and 4D contrast imaging systems may include an ultrasound machine for acquiring image data. The system may also include a 3D/4D processor for converting the image data into one or more contrast-enhanced images that are one or more of a 3D image and a 4D image. In addition, the system may include one or more displays, adapted to display i) one or more contrast-enhanced images, ii) one or more reconstructed CTI images using a CTI processor, and/or iii) one or more TIC curves using a TIC processor. The system may also include one or more storage servers capable of storing image data, wherein the image data includes the contrast-enhanced image(s), CTI image data, TIC image date, reconstructed CTI image(s), and/or TIC curve(s).

In additional embodiments, methods for applying contrast imaging on 3D and 4D images include converting image data acquired by an ultrasound machine into one or more contrast-enhanced images that are a 3D image and/or a 4D image. The method may also include generating one or more reconstructed CTI images based on the one or more contrast-enhanced images using a CTI processor and/or one or more TIC curves based on the one or more contrast-enhanced images using a TIC processor. In addition, the method may also include storing in one or more storage servers the contrast-enhanced image(s), the reconstructed CTI image(s), and/or the TIC curve(s). The method may also include displaying the contrast-enhance image(s), the reconstructed CTI image(s), and/or the TIC curve(s).

And yet other embodiments include a computer-readable storage medium including a set of instructions for a computer. In certain embodiments, the set of instructions include an image conversion routine for converting image data acquired by an ultrasound machine into one or more contrast-enhanced images that are a 3D image and/or a 4D image. The set of instructions may also include a reconstruction routine for reconstructing one or more CTI images based on the one or more contrast-enhanced images using a CTI processor. In addition, the set of instructions may also include a generation routine for generating one or more TIC curves based on the one or more contrast-enhanced images using a TIC processor. The set of instructions may also include a storage routine for storing the contrast-enhanced image(s), the CTI image(s), and/or the TIC curve(s). The set of instructions may also include a display routine for displaying the contrast-enhanced image(s), the CTI image(s), and/or the TIC curve(s).

### BRIEF DESCRIPTION OF SEVERAL VIEWS OF THE DRAWINGS

FIG. 1 illustrates a 3D and 4D contrast imaging system used in accordance with an embodiment of the present invention.

FIG. 2 illustrates an example of contrast tomography imaging in time plane display. The illustration displays the same image plane in each volume in time sequence.

FIG. 3 illustrates an example of contrast tomography imaging in time volume display. The illustration displays a volume image formed by combining the same image plane in all volumes in time sequence.

FIG. 4 illustrates an example of a graph displaying the average contrast intensity in an area or volume as a function of time.

5 illustrates contrast tomography imaging in space. The illustration displays multiple image planes in a volume.

6 illustrates a flow diagram for a method for applying contrast imaging on 3D and 4D images using ultrasound technology in accordance with an embodiment of the present invention.

The foregoing summary, as well as the following detailed description of certain embodiments of the present invention, will be better understood when read in conjunction with the appended drawings. For the purpose of illustrating the invention, certain embodiments are shown in the drawings. It should be understood, however, that the present invention is not limited to the arrangements and instrumentality shown in the attached drawings.

### DETAILED DESCRIPTION OF VARIOUS PREFERRED EMBODIMENTS

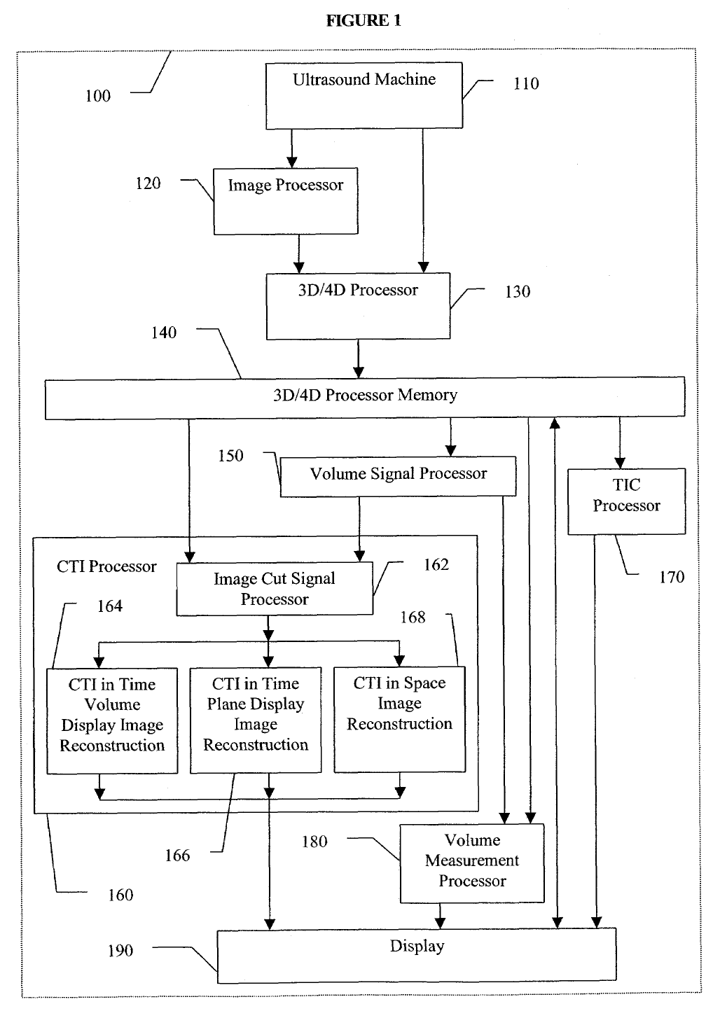

FIG. 1 illustrates a 3D and 4D contrast imaging system 100 used in accordance with an embodiment of the present invention. The system 100 includes an ultrasound machine 110, an image processor 120 and/or a 3D/4D processor 130, a 3D/4D processor memory 140, a volume signal processor 150, a contrast tomography imaging (CTI) processor 160, a time intensity curve (TIC) processor 170, a volume measurement processor 180, and a display 190. The CTI processor 160 may include an image cut signal process level component 162, a CTI in time volume display image reconstruction component 164, a CTI in time plane display image reconstruction component 166, and/or a CTI in space image reconstruction component 168, among other things. The components of the system 100 may communicate via wired and/or wireless connections on one or more processing units, such as computers, medical systems, storage devices, custom processors, and/or other processing units. The system 100 may be implemented in software and/or hardware. In certain embodiments, the 3D and 4D contrast imaging system 100 is integrated into a single unit, or may be integrated in various forms. In an embodiment, a user of the system may be a doctor, sonographer, medical practitioner, technician, other hospital staff member, or the like. Accordingly, the technical effect is an enhanced diagnostic arrangement using contrast agents in 3d and/or 4D ultrasound imaging.

The system 100 may be used to provide a solution for applying contrast imaging on 3D and 4D images using ultrasound technology to improve the accuracy of a diagnosis and the efficiency in which a medical practitioner and/or technician is able to view a patient's images based on a patient's images from previous examinations. A sonographer, medical practitioner, technician, or other hospital staff member that performs ultrasound examinations often uses 3D and/or 4D images. These images can offer views of the anatomy that assist a doctor in visualizing and detecting abnormalities. The use of a contrast agent injected in to the body while acquiring 3D and/or 4D images presents a significant clinical benefit by assisting a doctor or medical practitioner in diagnosing patient abnormalities. For example, with contrast imaging, blood flow in very small blood vessels becomes visible. Monitoring contrast enhancement change as a function of time can provide important diagnostic information, for example, in tumor identification and classification. In an embodiment, examples of 3D and/or 4D images include: multiplanar images, curve planar images, volume rendered images, fly-through image views, fly-around image views, maximum intensity projections, and minimum intensity projections, among others. Saggital and coronal views, among others, are examples of multiplanar image views in accordance with an embodiment of the present invention. One skilled in the art would recognize that there are many types of 3D and/or 4D images and subsets of views there from. The types of 3D and 4D images and any views mentioned are for exemplary purposes and are not intended to limit the present invention.

In an embodiment, the ultrasound machine 110 acquires a 3D or 4D image, by electronically, mechanically, and/or manually controlling an ultrasound transducer to sweep a volume image of a region of interest. Acquiring a single volume forms a 3D image. Acquiring the multiple volume image within a certain amount of time forms a 4D image. If the image is being acquired electronically or mechanically, a user may use, for example, the display 190 to enter information used by the ultrasound transducer to perform the ultrasound examination. For example, when performing an electronic or mechanical ultrasound examination using the display 190 to control the ultrasound transducer, a user may use the display to determine one or more of the following: a region of interest, an angle of an acquired volume, a quality of acquisition (dictated by the time of acquisition), a primary plane of acquisition (e.g., longitudinal or transverse), and whether a 3D or 4D image will be acquired, among other things. In an embodiment, while performing an ultrasound examination using contrast, when contrast agents enter the artery, the probe sweep direction may line up with the blood flow direction to get the contrast image of the whole blood vessel. In 4D contrast imaging, at least in the early phase, the probe may sweep unidirectionally so that the user has control of the sweep direction relative to the blood flow.

In an embodiment, the display 190 is used by a user to, among other things, view and interact with images created as a result of an ultrasound examination and, if electronically controlling the ultrasound machine 110, the display 190 may be used to perform the ultrasound examination. An image from an examination may be displayed on an image viewer, the image viewer being a program or application on (or accessible to) the display 190. In an embodiment, a 3D and/or 4D image can be displayed as sectional images (i.e., viewing each of the three perpendicular 3D/4D planes independently in 2D) and/or as a 3D/4D volume rendered image, among other things. While viewing the image from the examination, a user may use one or more applications associated with the volume signal processor 150, CTI processor 160, TIC processor 170, and/or volume measurement processor 180, among other things, for example, to alter the views of the image, filter out image information in order to locate possible abnormalities in the image, and/or graph a time intensity curve 400 (if a 4D image). The one or more applications associated with the volume signal processor 150, CTI processor 160, TIC processor 170, and/or volume measurement processor 180, which are programs or applications on (or accessible to) the display 190 may be integrated with the image viewer or a separate application. In an embodiment, the display 190, may also contain the image processor 120 and/or 3D/4D processor 130, 3D/4D processor memory 140, the volume signal processor 150, the CTI processor 160, the TIC processor 170, and/or the volume measurement processor 180, among other things. The image processor 120 and/or 3D/4D processor 130, volume signal processor 150, CTI processor 160, TIC processor 170, and volume measurement processor 180 may be a set of commands, which may be implemented in software and/or hardware on the 3D/4D processor memory 140 or display 190, such as a PACS workstation, or other workstation. In an embodiment, a sonographer or other user of the system 100 may view and alter previous examinations saved in the 3D/4D processor memory 140. In an embodiment, the display 190 may be a touch panel display, voice controlled display, a displayed used with buttons, knobs, or the like, and/or a display used with a keyboard/mouse (e.g., computer), among other things.

In an embodiment, the image processor 120 is used to convert image data from the ultrasound machine 110 into a 2D image. The 3D/4D processor 130 is used to convert image data from the ultrasound machine 110 or image processor 120 into a 3D or 4D image. In an embodiment, the image processor 120 converts the image data from the ultrasound machine 110 into a 2D image before sending the 2D image data to the processor memory (that may be the 3D/4D processor memory 140 or a separate memory) and/or the 3D/4D processor 130 to convert into a 3D or 4D image. In another embodiment, the ultrasound machine 110 sends image data directly to the 3D/4D processor 130. In yet another embodiment, the image processor 120 and 3D/4D processor 130 may be integrated in one component capable of converting image data from the ultrasound machine 110 into 2D, 3D and/or 4D images.

In an embodiment, a 3D/4D processor memory 140 is used to save images created from an examination after a sonographer or other healthcare professional performs an ultrasound examination on a patient and the image data is converted by the image processor 120 and/or 3D/4D processor 130. Further, parameters for creating or altering ultrasound examination images, which may be created by a sonographer or other healthcare professional when reconstructing 3D or 4D images, creating a TIC curve 500, or rendering 3D or 4D images, may be saved to the 3D/4D processor memory 140. Alternatively or in addition, the 3D/4D images may be stored on the display 190 and/or on the 3D/4D processor memory 140, which may be a PACS server, a database, a library, or other general memory, among other things. The 3D/4D processor memory 140 may also store one or more of the image processor 120 and/or 3D/4D processor 130, CTI processor 160, TIC processor 170, and/or volume measurement processor 180, among other things. The 3D/4D processor memory 140 may be integrated with the display 190 or be a separate system. In an embodiment, images saved in a current examination by the 3D/4D processor 130 are displayed on the display 190. In another embodiment, saved images from current and/or previous examinations are sent to one or more of the CTI processor 160, TIC processor 170, and/or volume measurement processor 180 as commanded by the display 190 at the instruction of a user of the system 100.

In an embodiment, the volume signal processor 150 is used to perform signal processing on a volume image (e.g., upon command from the display 190 at the direction of a user). The volume signal processor 150 uses signal processing to enhance the image volume and allows a user to get the maximum amount of information from the data. In an embodiment, a user may use the volume signal processor 150 to smooth or enhance surface texture, minimize or maximize transparency, and increase or decrease gradient light on the rendered volume image, among other things. For example, a user may use the volume signal processor 150 to maximize the transparency of a volume to more easily view dense matter in an image volume, improving a sonographer's or doctor's ability to diagnose a patient.

In an embodiment, the 3D and 4D contrast imaging system 100 uses a CTI processor 160 to perform signal processing on an image 162 and/or to reconstruct a 4D image in time volume display 164, time plane display 166, and/or to reconstruct a 3D or 4D image in space display 168. For example, a sonographer who has performed an ultrasound examination on a patient may want to view a CTI image reconstructed in time plane display 200, a CTI image reconstructed in time volume display 300, and/or a CTI image reconstructed in space display 400.

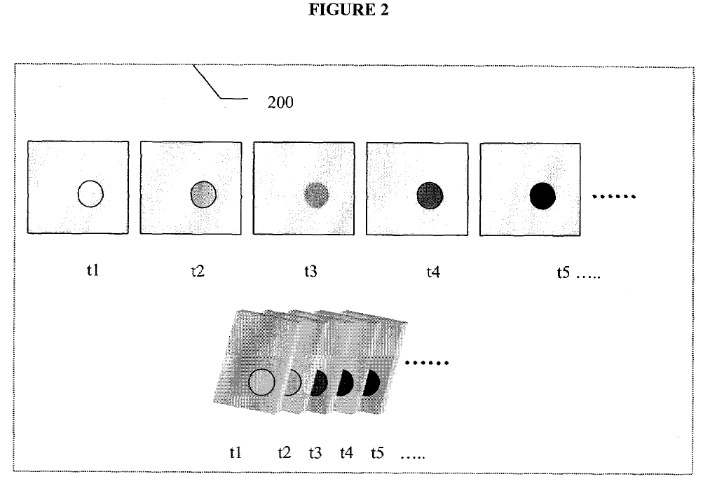

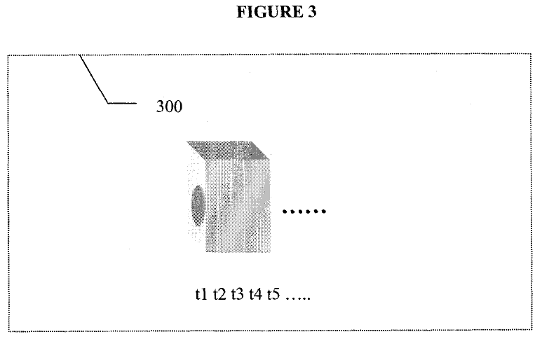

FIG. 2 illustrates a CTI image in time plane display 200. A CTI image in time plane display 200 displays the same image plane in each user selected volume or in all the volumes in time sequence. The image plane can be selected in any orientation and position in a volume. If there too many images to display at once, user can use a knob or a key to scroll through all the images. A CTI image in time plane display 200 can be reconstructed using a 4D image (or multiple 3D images over a period of time) because the image 200 is over a period of time. FIG. 3 illustrates a CTI image in time volume display 300. A CTI image in time volume display 300 displays a volume image formed by combining the same image plane in all volumes in time sequence. A CTI image in time volume display 300 can be reconstructed using a 4D image (or multiple 3D images over a period of time) because the image 300 is over a period of time. CTI images in time plane display 200 and time volume display 300 may be useful for a doctor or sonographer because the doctor or sonographer may monitor the contrast enhancement change as a function of time, which may assist in, for example, tumor identification and classification.

FIG. 5 illustrates a CTI image in space display 500. A CTI image in space display 500 displays multiple image planes inside a volume. A CTI image in space display 500 can be reconstructed using either a 3D image or one volume image from a 4D image. A CTI image in space can be formed along any orientation in the volume and the image planes in the CTI are evenly spaced. The spacing between the image planes are selectable by user. If too many image planes are to be displayed, a knob or a key can be used to scroll through the image planes. The original examination images and subsequent reconstructed images 200, 300, 500 may be stored on the 3D/4D processor memory 140 or a separate storage server (e.g., a storage server integrated with the display 190).

In an embodiment, the CTI processor 160 uses an image cut signal processor 162 to perform signal processing on an image cut (preferably upon command from the display 190 at the direction of a user). The image cut signal processor 162 uses signal processing to enhance the image cuts (or planes) and allows a user to get the maximum amount of information from the data. In an embodiment, a user may use the image cut signal processor 162 to smooth an area of interest within an image cut or enhance the boundary (or structure) of an area of interest within an image cut, among other things. For example, a user may use the image cut signal processor 162 to smooth within a very thin slice around each image plane to improve the image quality. The image cut signal processor 162 may receive an original 3D or 4D image from the 3D/4D processor memory 140 to perform image cut signal processing on or the 3D or 4D image may be an image already enhanced by the volume signal processor 150 that needs further enhancement at the image cut level before image reconstruction by another component of the CTI processor 164-168. If a user determines no image cut signal processing is needed, the image data is sent directly to the CTI image reconstruction component 164-168 selected by the user of the system 100.

In an embodiment, upon a command from the display 190 at the instruction of a user, the 3D and 4D contrast imaging system 100 reconstructs a 4D image in time volume display 164, time plane display 166, and/or reconstructs a 3D or 4D image in space display 168 by performing an algorithm on the 4D image data (or in the case of CTI in space display 168, performing an algorithm on the 3D image data or on a moment in time of the 4D image data). The image data may be sent to the CTI processor 160 via the 3D/4D processor memory 140 and may be altered by the image cut signal processor 162 at the discretion of the user of the system 100 (depending on if the user instructs the system 100 to perform signal processing to improve the image quality). In an embodiment, after the image data is reconstructed into a CTI image in time volume display 300, CTI image in time plane display 200, or CTI image in space display 500, the reconstructed image 200, 300, 500 is sent to and displayed upon the display 190. In another embodiment, the reconstructed CTI images can also be output as one or more data files. In an embodiment, the data files and/or reconstructed images 200, 300, 500 may be stored on the 3D/4D processor memory 140 or a separate storage server (e.g., a storage server integrated with the display 190).

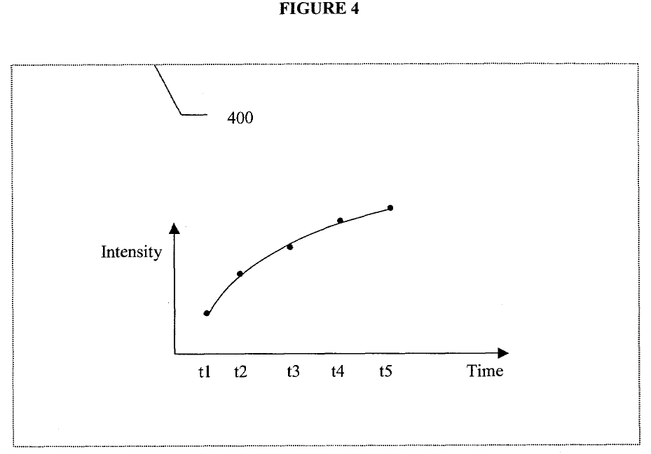

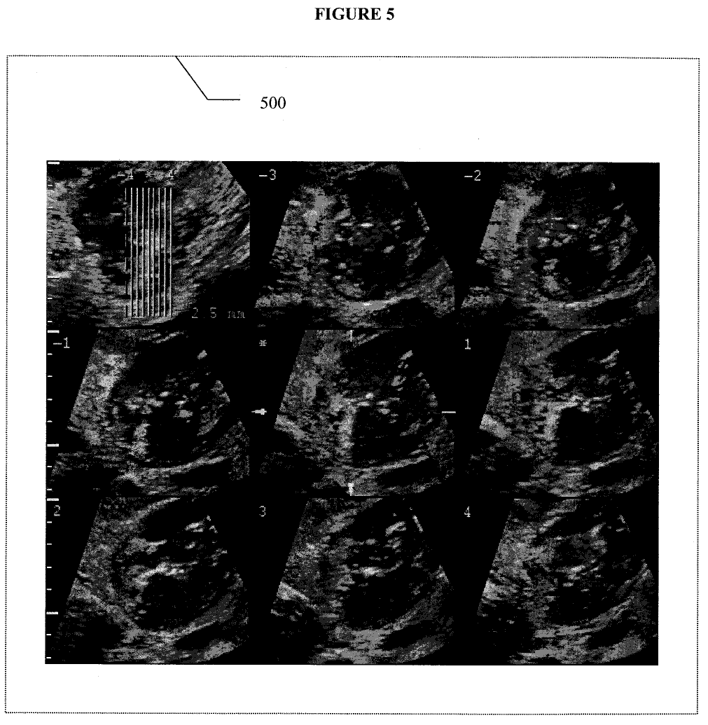

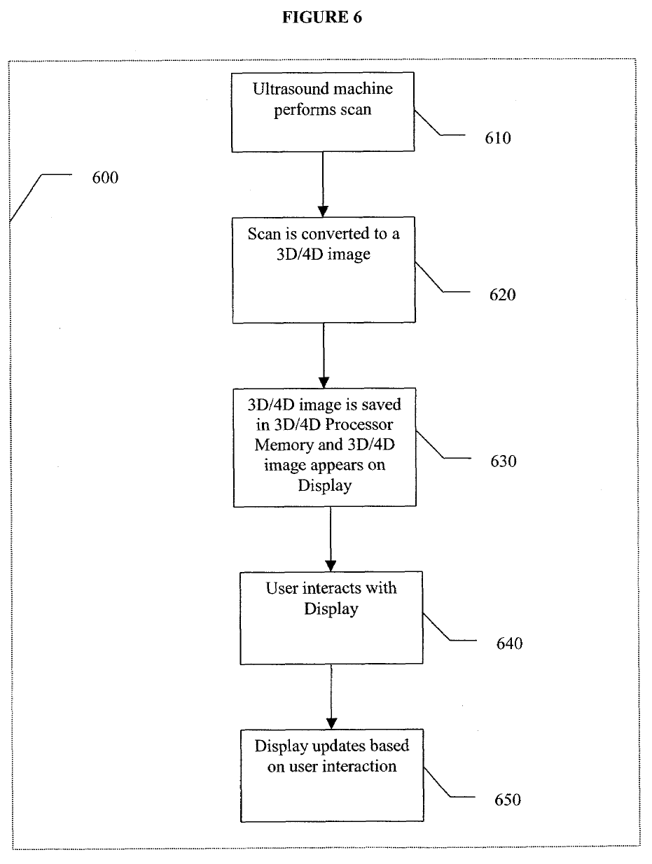

In an embodiment, a TIC processor 170 is used to graph the average contrast intensity in an area or a volume as a function of time 400. FIG. 4 illustrates an example TIC curve 400. In an embodiment, upon a command from the display 190 at the instruction of a user, the 3D and 4D contrast imaging system 100 measures the averaged contrast intensity inside a user selected area or volume, calculates the intensity in each volume image, and plots the intensity curve as a function of time (TIC curve) 400 for future analysis by performing an algorithm on the 4D image data. In another embodiment, the TIC curve 400 can also be output as a data file, for example as intensity and time table, among other things. The image data may be sent to the TIC processor 170 via the 3D/4D processor memory 140. In an embodiment, after the image data is converted to a graph of the TIC curve 400, the graph 400 is sent to and displayed upon the display 190. In an embodiment, the graph of the TIC curve 400 and/or the data file created by the TIC processor 170 may be stored on the 3D/4D processor memory 140 or a separate storage server (e.g., a storage server integrated with the display 190).

In an embodiment, a volume measurement processor 180 is used to view areas of interest within an image volume. For example, if after viewing a 3D or 4D image from an ultrasound examination, a user wants to measure the volume on a tumor within the volume image, the user may use the display 190 to outline the boundary of the tumor and using the volume measurement processor 180 to measure the tumor. The user can then use the display 190 to rotate and view the tumor structure at any angle. In another embodiment, the display 190 and/or volume measurement processor 180 may automatically detect the boundary of a tumor within the volume image to display the tumor and measure its volume. In yet another embodiment, the user may outline the tumor boundary from one or two of the section plans (a 3D/4D image can be displayed in sectional format in A, B and C three perpendicular planes) display 190 and/or volume measurement processor 180 automatically detects the boundary in the rest sectional plans.

In operation, a patient injected with contrast agent is scanned mechanically, manually or electronically using an ultrasound machine 110. The ultrasound machine 110 sends image data to the image processor 120, where the image data is converted into a 2D image. The image data from the image processor 120 is then sent to the 3D/4D processor 130, where the 2D image data is converted into 3D or 4D image data. Alternatively, the image data from the ultrasound machine 110 may be sent directly to the 3D/4D processor 130, where the image data from the ultrasound examination is converted into a 3D or 4D image. The 3D or 4D image is then saved in the 3D/4D processor memory 140 and displayed on the display 190. A user views the image on the display 190 using an image viewer. The user uses the volume signal processor 150, the CTI processor 160, the TIC processor 170 and/or the volume measurement processor 180 to view and diagnose a patient. If using the CTI processor 160 or the volume measurement processor 180, the sonographer or other medical practitioner may choose to perform volume signal processing on the volume image using the volume signal processor 150 to improve image quality of the volume image. If using the CTI processor 160, the sonographer or other healthcare professional may perform image plane signal processing on one or more image planes using the image cut signal processor 162 to improve the quality of one or more image cuts. Additionally, if using the CTI processor 160, the sonographer or other healthcare professional may choose to view CTI image reconstruction in time volume display (if a 4D image) 166, time plane display (if a 4D image) 164, and/or space display (for either a 3D image or a moment in time of a 4D image) 168. The reconstructed image 200, 300, 500 (generated from the CTI processor 160), TIC curve 400 (generated from the TIC processor 170), or reconstructed volume measurement image (generated from the volume measurement processor 180) is saved to the 3D/4D processor memory 140 with any parameters used to create the reconstructed image 200, 300, 500, TIC curve 400, or reconstructed volume measurement image. At any time thereafter, a sonographer or medical practitioner with access to the system 100 may retrieve saved images or graphs from the 3D/4D processor memory 140 to further analyze and/or to create additional images using the CTI processor 160, TIC processor 170, and/or volume measurement processor 180.

FIG. 6 illustrates a flow diagram 600 for a method for applying contrast imaging on 3D and 4D images using ultrasound technology in accordance with an embodiment of the present invention.

First, at step 610, an ultrasound examination is performed by a sonographer or other healthcare professional that results in an image or group of images being created. For example, a sonographer may perform an ultrasound examination either by electronically, mechanically, and/or manually controlling the ultrasound transducer to sweep a volume image of the region of interest. Acquiring a single volume forms a 3D image. Acquiring the multiple volume image within a certain amount of time forms a 4D image. If the image is being acquired electronically or mechanically, a user may use, for example, the display 190 to enter information used by the ultrasound transducer to perform the ultrasound examination. For example, when performing an electronic or mechanical ultrasound examination using the display 190 to control the ultrasound machine 110, a user may use the display to determine one or more of the following: a region of interest, an angle of an acquired volume, a quality of acquisition (dictated by the time of acquisition), a primary plane of acquisition (e.g., longitudinal or transverse), and whether a 3D or 4D image will be acquired, among other things. In an embodiment, while performing an ultrasound examination using contrast, when contrast agents enter the artery, the probe sweep direction may line up with the blood flow direction to get the contrast image of the whole blood vessel. In 4D contrast imaging, at least in the early phase, the probe may sweep unidirectionally so that the user has control of the sweep direction relative to the blood flow.

At step 620, the image data acquired from the ultrasound machine 110 is converted to a 3D or 4D image. In an embodiment, the image processor 120 is used to convert image data from the ultrasound machine 110 into a 2D image. The 3D/4D processor 130 is used to convert image data from the ultrasound machine 110 or image processor 120 into a 3D or 4D image. In an embodiment, the image processor 120 converts the image data from the ultrasound machine 110 into a 2D image before sending the 2D image data to the processor memory (that may be the 3D/4D processor memory 140 or a separate memory) and/or the 3D/4D processor 130 to convert into a 3D or 4D image. In another embodiment, the ultrasound machine 110 sends image data directly to the 3D/4D processor 130. In yet another embodiment, the image processor 120 and 3D/4D processor 130 may be integrated in one component capable of converting image data from the ultrasound machine 110 into 2D, 3D and/or 4D images.

Then, at step 630, the 3D or 4D image is saved on the 3D/4D processor memory 140 and the image is displayed on the display 190. In an embodiment, a 3D/4D processor memory 140 is used to save images created from an examination after a user performs an ultrasound examination on a patient and the image data is converted by the image processor 120 and/or 3D/4D processor 130. Further, parameters for creating or altering ultrasound examination images, which may be created by a user when reconstructing 3D or 4D images, creating a TIC curve 500, and/or rendering 3D or 4D images, may be saved to the 3D/4D processor memory 140. Alternatively or in addition, the 3D/4D images may be stored on the display 190 and/or on the 3D/4D processor memory 140, which may be a PACS server, a database, a library, or other general memory, among other things. The 3D/4D processor memory 140 may also store one or more of the image processor 120 and/or 3D/4D processor 130, CTI processor 160, TIC processor 170, and/or volume measurement processor 180, among other things. The 3D/4D processor memory 140 may be integrated with the display 190 or be a separate system. In an embodiment, images saved in a current examination by the 3D/4D processor 140 are displayed on the display 190. In another embodiment, saved images from current and/or previous examinations are sent to one or more of the CTI processor, TIC processor and/or volume measurement processor as commanded by the display 190 at the instruction of a user of the system 100.

Next, at step 640, the user interacts with the display 190. The user using the system 100 may view the original image from the ultrasound examination by interacting with the display 190 to rotate the image and zoom in or out on various structures within the image, among other things. If viewing a 4D image, a user may also go through the 4D image volume by volume (over a period of time) using a key or knob, for example, associated with the display 190. Additionally, a user may perform various functions on the original 3D or 4D image to easily, quickly and effectively diagnose a patient.

For example, a user may use the volume signal processor 150 to enhance the image volume and allows a user to get the maximum amount of information from the data. In an embodiment, a user may use the volume signal processor 150 to smooth or enhance surface texture, minimize or maximize transparency, and increase or decrease gradient light, among other things. For example, a user may use the volume signal processor 150 to maximize the transparency of a volume to more easily view dense matter in an rendered volume image, improving a sonographer's or doctor's ability to diagnose a patient.

In another example, a user may use the TIC processor 170 to graph the average contrast intensity in an area or a volume as a function of time 400. Upon a command from the display 190 at the instruction of a user, the 3D and 4D contrast imaging system 100 measures the averaged contrast intensity inside a user selected area or volume, calculates the intensity in each volume image, and plots the intensity curve as a function of time (TIC curve) 400 for future analysis by performing an algorithm on the 4D image data. The image data may be sent to the TIC processor 170 via the 3D/4D processor memory 140. In an embodiment, after the image data is converted to a graph of the TIC curve 400, the graph 400 is sent to and displayed upon the display 190. In an embodiment, the graph of the TIC curve 400 created by the TIC processor 170 may be stored on the 3D/4D processor 140 or a separate storage server (e.g., a storage server integrated with the display 190).

A user may also use the volume measurement processor 180 to view areas of interest within an image volume. For example, if after viewing a 3D or 4D image from an ultrasound examination, a user wants to view a particular structure within the volume image by itself, the user may use the display 190 to select the boundaries of the structure and using the volume measurement processor 180, the display 190 will display the 3D or 4D image structure by itself. The user can then use the display 190 to rotate the image structure and view the image structure at any angle. In another embodiment, the display 190 and/or volume measurement processor 180 may automatically detect the boundaries of a structure within the volume image to display. In yet another embodiment, the user may outline the tumor boundary from one or two of the three sectional planes and the display 190 and/or volume measurement processor 180 automatically detects the boundary in the remaining sectional planes and calculates the tumor volume.

In another example, a sonographer or other medical practitioner may use the CTI processor 160 to perform signal processing on an image 162 and/or to reconstruct a 4D image in time volume display 164, time plane display 166, and/or to reconstruct a 3D or 4D image in space display 168. For example, a sonographer who has performed an ultrasound examination on a patient may want to view a CTI image reconstructed in time plane display 200, a CTI image reconstructed in time volume display 300, and/or a CTI image reconstructed in space display 400. Upon a command from the display 190 at the instruction of a user, the 3D and 4D contrast imaging system 100 reconstructs a 4D image in time volume display 164, time plane display 166, and/or reconstructs a 3D or 4D image in space display 168 by performing an algorithm on the 4D image data (or in the case of CTI in space display 168, performing an algorithm on the 3D image data or on a moment in time of the 4D image data). The image data may be sent to the CTI processor 160 via the 3D/4D processor memory 140 and may be altered by the image cut signal processor 162 at the discretion of the sonographer or other user of the system 100 (depending on if the user instructs the system 100 to perform signal processing to improve the image quality).

Then, at step 650, the display updates based on the commands given to the system 100 by a user (e.g., sonographer or other medical practitioner). If a TIC curve or reconstructed image was created by the sonographer or other medical practitioner, those TIC curves, reconstructed images, and any parameters used to create the TIC curves and/or reconstructed images may be saved to the 3D/4D processor memory, the display 190, and/or another storage server. For example, after the image data is reconstructed into a CTI image in time volume display 300, CTI image in time plane display 200, or CTI image in space display 500, the reconstructed image 200, 300, 500 is sent to and displayed upon the display 190. The reconstructed images 200, 300, 500 may also be stored on the 3D/4D processor 140 or a separate storage server (e.g., a storage server integrated with the display 190).

Thus, certain embodiments accelerate and increased the productivity of a doctor, sonographer, or other medical practitioner's ability to view and diagnose abnormalities in a patient's ultrasound examination. Increased productivity includes a speed in which a diagnosis may be performed and an accuracy of reports produced based on the diagnosis.

While the invention has been described with reference to certain embodiments, it will be understood by those skilled in the art that various changes may be made and equivalents may be substituted without departing from the scope of the invention. In addition, many modifications may be made to adapt a particular situation or material to the teachings of the invention without departing from its scope. Therefore, it is intended that the invention not be limited to the particular embodiment disclosed, but that the invention will include all embodiments falling within the scope of the appended claims.

What is claimed is:

1. An ultrasound imaging system, comprising:
an ultrasound machine for acquiring ultrasound image data; and
a processor for converting the ultrasound image data into a contrast-enhanced three-dimensional (3D) image.

2. The ultrasound imaging system of claim 1, further comprising a contrast tomography imaging (CTI) processor.

3. The ultrasound imaging system of claim 2, wherein the CTI processor is adapted to generate CTI image data.

4. The ultrasound imaging system of claim 1, further comprising a time intensity curve (TIC) processor.

5. The ultrasound imaging system of claim 4, wherein the TIC processor is adapted to generate TIC image data.

6. An ultrasound imaging system, comprising:
an ultrasound machine for acquiring ultrasound image data; and
a processor for converting the ultrasound image data into a contrast-enhanced four-dimensional (4D) image.

7. The ultrasound imaging system of claim 6, further comprising a contrast tomography imaging (CTI) processor.

8. The ultrasound imaging system of claim 7, wherein the CTI processor is adapted to generate CTI image data.

9. The ultrasound imaging system of claim 6, further comprising a time intensity curve (TIC) processor.

10. The ultrasound imaging system of claim 9, wherein the TIC processor is adapted to generate TIC image data.

11. A method for acquiring an ultrasound image, comprising:
providing an ultrasound machine for acquiring ultrasound image data; and
providing a processor for converting the ultrasound image data into a three-dimensional (3D) contrast-enhanced image.

12. The method of claim 11, further comprising providing a contrast tomography imaging (CTI) processor.

13. The method of claim 12, wherein the CTI processor is adapted to generate CTI image data.

14. The method of claim 11, further comprising providing a time intensity curve (TIC) processor.

15. The method of claim 14, wherein the TIC processor is adapted to generate TIC image data.

16. A method for acquiring an ultrasound image, comprising:
providing an ultrasound machine for acquiring ultrasound image data; and
providing a processor for converting the ultrasound image data into a four-dimensional (4D) contrast-enhanced image.

17. The method of claim 16, further comprising providing a contrast tomography imaging (CTI) processor.

18. The method of claim 17, wherein the CTI processor is adapted to generate CTI image data.

19. The method of claim 16, further comprising providing a time intensity curve (TIC) processor.

20. The method of claim 19, wherein the TIC processor is adapted to generate TIC image data.

21. A method for acquiring a three-dimensional (3D) contrast-enhanced ultrasound image, comprising:
providing a contrast-enhancing agent;
providing an ultrasound machine for acquiring ultrasound image data; and
providing a processor for converting the ultrasound image data into a three-dimensional (3D) contrast-enhanced image using the contrast-enhancing agent.

22. A method for acquiring a four-dimensional (4D) contrast-enhanced ultrasound image, comprising:
providing a contrast-enhancing agent;
providing an ultrasound machine for acquiring ultrasound image data; and
providing a processor for converting the ultrasound image data into a four-dimensional (4D) contrast-enhanced image using the contrast-enhancing agent.

23. A three-dimensional (3D) and four-dimensional (4D) contrast imaging system, comprising:
an ultrasound machine for acquiring ultrasound image data;
a 3D/4D processor for converting the ultrasound image data into at least one contrast-enhanced image that is at least one of:
a 3D image, and
a 4D image;
at least one contrast tomography imaging (CTI) processor, adapted to generate CTI image data; and
at least one time intensity curve (TIC) processor, adapted to generate TIC image data.

24. The system of claim 23, further comprising at least one display, adapted to:
display the at least one contrast-enhanced image, and
display at least one of:
at least one reconstructed CTI image using the at least one CTI processor, and
at least one TIC curve using the at least one TIC processor.

25. The system of claim 24, further comprising at least one storage server capable of storing at least one of:
the at least one contrast-enhanced image,
the CTI image data,
the TIC image data,
the at least one reconstructed CTI image, and
the at least one TIC curve.

26. The system of claim 24, wherein at least one of:
the at least one contrast-enhanced image,
the at least one reconstructed CTI image, and
the at least one TIC curve, is displayed on the at least one display using an image viewer.

27. The system of claim 23, further comprising a volume signal processor for performing signal processing on at least one contrast-enhanced image volume.

28. The system of claim 24, wherein the CTI image data is at least one of:
the at least one reconstructed CTI image, and
a file containing data to create the at least one reconstructed CTI image.

29. The system of claim 23, further comprising a volume measurement processor for viewing a user selected structure within at least one contrast-enhanced image volume.

30. The system of claim 24, wherein the at least one reconstructed CTI image is at least one of:
a CTI image in time volume display created from the 4D image,
a CTI image in time plane display created from the 4D image, and
a CTI image in space display created in at least one of:
the 3D image, and
one volume from the 4D image.

31. The system of claim 30, wherein an image cut signal processor performs signal processing on the at least one reconstructed CTI image.

32. The system of claim 24, wherein TIC curve data is at least one of:
the at least one TIC curve, and
a file containing data to create the at least one TIC curve.

33. The system of claim 23, wherein the ultrasound machine uses a transducer probe that sweeps unidirectionally to enable control of sweep direction relative to blood flow when acquiring the ultrasound image data.

34. A method for applying contrast imaging on three-dimensional (3D) and four-dimensional (4D) images, the method comprising:
converting image data acquired by an ultrasound machine into at least one contrast-enhanced image that is at least one of:
a 3D image, and
a 4D image; and
generating at least one of:
at least one reconstructed contrast tomography imaging (CTI) image based on the at least one contrast-enhanced image using a CTI processor, and
at least one time intensity curve (TIC) curve based on the at least one contrast-enhanced image using a TIC processor.

35. The method of claim 34, further comprising storing in at least one storage server at least one of:
the at least one contrast-enhanced image,
the at least one reconstructed CTI image, and
the at least one TIC curve.

36. The method of claim 34, further comprising displaying at least one of:
the at least one contrast-enhanced image,
the at least one reconstructed CTI image, and
the at least one TIC curve.

37. The method of claim 34, wherein the at least one reconstructed CTI image includes at least one of:
at least one CTI image in time volume display,
at least one CTI image in time plane display, and
at least one CTI image in space display.

38. The method of claim 34, further comprising enhancing the at least one contrast-enhanced image by performing signal processing on at least one image plane of the at least one contrast-enhanced image using an image cut processor.

39. The method of claim 36, further comprising constructing a volume measurement image using a volume measurement processor for viewing a selected structure within an image volume.

40. The method of claim 39, wherein the selected structure is identified by at least one of:
manual selection of structure boundaries by a user,
automatic detection of structure boundaries, and
manual selection of at least one structure boundary by the user and automatic detection of at least one remaining structure boundary.

41. The method of claim 34, further comprising improving the at least one contrast-enhanced image by performing signal processing on at least one contrast-enhanced image volume using a volume signal processor.

42. A computer-readable storage medium including a set of instructions for a computer, the set of instructions comprising:
an image conversion routine for converting image data acquired by an ultrasound machine into at least one contrast-enhanced image that is at least one of:
a three-dimensional (3D) image, and
a four-dimensional (4D) image;
a reconstruction routine for reconstructing at least one contrast tomography imaging (CTI) image based on the at least one contrast-enhanced image using a CTI processor; and
a generation routine for generating at least one time intensity curve (TIC) curve based on the at least one contrast-enhanced image using a TIC processor.

43. The computer-readable storage medium of claim 42, wherein the set of instructions further comprises a storage routine for storing at least one of:
the at least one contrast-enhanced image,
the at least one CTI image, and
the at least one TIC curve.

44. The computer-readable storage medium of claim 42, wherein the set of instructions further comprises a display routine for displaying at least one of:
the at least one contrast-enhanced image,
the at least one CTI image, and
the at least one TIC curve.

45. The computer-readable storage medium of claim 42, wherein the CTI processor includes at least one routine for at least one of:
reconstructing the at least one CTI image in time volume display,
reconstructing the at least one CTI image in time plane display, and
reconstructing the at least one CTI image in space display.

46. The computer-readable storage medium of claim 42, wherein the set of instructions further comprises an image cut signal processing routine that performs signal processing on at least one image plane of the at least one contrast-enhanced image.
